(19) United States
(12) Patent Application Publication
(10) Pub. No.: US 2023/0172847 A1
(43) Pub. Date: Jun. 8, 2023

(54) PROCESS FOR PRODUCING AN ORAL THIN FILM COMPRISING MICROPARTICLES

(71) Applicant: LTS LOHMANN THERAPIE-SYSTEM AG, Andernach (DE)

(72) Inventors: MICHAEL LINN, Waldböckelheim (DE); Markus MÜLLER, Troisdorf (DE); Marius BAUER, Andernach (DE)

(21) Appl. No.: 17/923,832

(22) PCT Filed: May 7, 2021
(86) PCT No.: PCT/EP2021/062098

§ 371 Date: Nov. 7, 2022

(30) Foreign Application Priority Data
May 7, 2020 (DE) 10 2020 112 422.8

(51) Int. Cl. A61K 9/00; A61K 47/34; A61K 47/38; A61K 31/122; A61K 31/192; A61K 9/14

(52) U.S. Cl. CPC A61K 9/006; A61K 47/34; A61K 47/38; A61K 31/122; A61K 31/192; A61K 9/141

(57) ABSTRACT

The present invention relates to a process for producing an oral thin film comprising microparticles, comprising the steps of:
a) producing a solution comprising a hydrophobic polymer, a hydrophobic pharmaceutically active ingredient and hydrophobic solvent,
b1) providing a hydrophilic polymer and providing a hydrophilic solvent immiscible with the solvent from a), or
b2) providing a solution comprising a hydrophilic polymer and a hydrophilic solvent immiscible with the solvent from a),
c1) mixing the solution from a) with the hydrophilic polymer from b1) followed by the addition of the hydrophilic solvent from b1) immiscible with the solvent from a) to obtain an emulsion, or
c2) mixing the solution from a) and the solution from b2) to obtain an emulsion, and
d) spreading and drying the emulsion from c1) or c2) to obtain an oral thin film comprising microparticles.

The present invention relates to a process for producing an oral thin film comprising microparticles, to an oral thin film obtainable by this process, and to the use of such an oral thin film as a medicament.

An oral thin film comprising microparticles usually contains microparticles that contain a pharmaceutically active ingredient, with these microparticles being embedded in a polymer matrix. This polymer matrix comprising the microparticles dissolves after administration of the oral thin film, and the microparticles comprising a pharmaceutically active ingredient are released and absorbed in the stomach and/or intestine of the patient after swallowing.

Oral thin films are known in the form of water-soluble films comprising an inner water-insoluble solid phase in the form of microparticles containing an active ingredient or flavouring.

To produce such films, microparticles comprising a hydrophobic polymer and a pharmaceutically active ingredient are usually prepared and isolated, as described for example in US 2015/0064231 A1, in order to be incorporated subsequently into a hydrophilic polymer matrix in a second process step.

However, such a production process has the disadvantage that at least two independent process steps are necessary to produce the oral thin film. Secondly, the separately produced microparticles have a relatively large particle size, which is also relatively difficult to adjust. A relatively large particle size of the microparticles comprising a hydrophobic polymer and a pharmaceutically active ingredient has the disadvantage that the bioavailability of the active ingredient is reduced.

The aim of the present invention is to overcome the above-mentioned disadvantages of the prior art. Especially, it is the aim of the present invention to provide a production process for an oral thin film comprising microparticles, by means of which process an oral thin film comprising microparticles can be produced with as few process steps as possible and as simply and economically as possible. In addition, the microparticles in the oral thin film produced by this process should have a particle size that is as small as possible, preferably smaller than 100 μm, preferably smaller than 50 μm, preferably smaller than 20 μm, especially preferably smaller than 10 μm, and very especially preferably smaller than 5 μm.

The above aim is addressed by the process according to claim 1, wherein the process comprises the following steps:

a) producing a solution comprising a hydrophobic polymer, a hydrophobic pharmaceutically active ingredient and hydrophobic solvent,
b1) providing a hydrophilic polymer and providing a hydrophilic solvent immiscible with the solvent from a), or
b2) providing a solution comprising a hydrophilic polymer and a hydrophilic solvent immiscible with the solvent from a),
c1) mixing the solution from a) with the hydrophilic polymer from b1) followed by the addition of the hydrophilic solvent from b1) immiscible with the solvent from a) to obtain an emulsion, or
c2) mixing the solution from a) and the solution from b2) to obtain an emulsion, and
d) spreading and drying the emulsion from c1) or c2) to obtain an oral thin film comprising microparticles.

With the one-step process according to the invention, the microparticles are not produced separately in advance and incorporated into a polymer matrix as in the known processes, but are produced by the production process due to the fact that two solutions with different hydrophilicity/hydrophobicity are mixed together. The components contained in solution a) form small droplets in the hydrophilic environment of the solution b) or b2) or the solvent from b1). The emulsion thus produced is then spread out and dried. During this drying process, the separation of the phases is maintained. Stable solid or semi-solid microparticles are formed, which may comprise the hydrophobic pharmaceutically active substance but also other constituents.

Preferably, the process according to the invention comprises the following steps:

a) producing a solution comprising a hydrophobic polymer, a hydrophobic pharmaceutically active ingredient and hydrophobic solvent,
b) providing a hydrophilic polymer and providing a hydrophilic solvent immiscible with the solvent from a),
c) mixing the solution from a) with the hydrophilic polymer from b) followed by the addition of the hydrophilic solvent from b) immiscible with the solvent from a) to obtain an emulsion, and
d) spreading and drying the emulsion from c) to obtain an oral thin film comprising microparticles.

Preferably, the process according to the invention comprises the following steps:

a) producing a solution comprising a hydrophobic polymer, a hydrophobic pharmaceutically active ingredient and hydrophobic solvent,
b) providing a solution comprising a hydrophilic polymer and a hydrophilic solvent immiscible with the solvent from a),
c) mixing the solution from a) and the solution from b) to obtain an emulsion, and
d) spreading and drying the emulsion from c) to obtain an oral thin film comprising microparticles.

Advantageously, the hydrophobic polymer can be chosen such that the release of the at least one hydrophobic pharmaceutically active ingredient can be controlled. For example, polymers can be chosen that do not dissolve in the acidic environment of the stomach, but only dissolve in the more basic environment of the intestine.

The term “comprise” can also mean “consisting of”.

The term “solution” also includes the term “suspension”.

The process according to the invention is preferably characterised in that the solvent in step b), b1) or b2) is water or an aqueous solvent.

Water as a solvent is especially preferred as it dissolves the polymers therein needed to form a film that is intended to dissolve at the application site where an aqueous environment is present. Also, a large difference in polarity is needed to prevent mixing of the two solvents, which is required for the formation of the two phases.

The hydrophobic solvent preferably comprises ethyl acetate, methyl acetate propyl acetate, liquid alkanes, liquid alkenes, aromatics, carboxylic acid esters, ethers and/or gasoline.

The solution from step b), b2) or the solvent provided in step b1) comprises a hydrophilic polymer. A hydrophilic polymer is a polymer containing polar and/or charged groups that render the polymer water-soluble.

The hydrophilic polymer can be selected from the group comprising starch and derivatives thereof, agar-agar, gelatin and other gel-forming proteins, cellulose and derivatives thereof, alginic acid, galactomannan, carrageenan and other vegetable gums, pullulan and other di- or polysaccharides), xanthan, pectin and other glucans, dextran, polyvinylpyrrolidone, polyvinyl alcohol, poly(meth)acrylates, polyalkylene glycols, carboxyvinyl polymers, polyethylene glycol-polyvinyl alcohol copolymers (obtainable for example under the trade name Kollicoat IR from BASF), shellac, polyvinyl caprolactam-polyvinyl acetate-polyethylene glycol graft and/or co-polymers thereof (obtainable for example under the trade name Soloplus from BASF) and/or co-polymers thereof, chitosan, polyoxyethylene alkyl ether.

In a preferred embodiment, the at least one hydrophilic polymer comprises polyvinylpyrrolidone, polyvinyl alcohol, a cellulose derivative and/or co-polymers thereof, polyethylene glycol-polyvinyl alcohol copolymers (obtainable for example under the trade name Kollicoat IR).

These hydrophilic polymers have the advantage that they form a thin stable film when dried, which dissolves upon application within a pharmaceutically acceptable period of time and releases the microparticles. This has the advantage of a relatively fast availability of the microparticles or the hydrophobic pharmaceutically active ingredient as well as a residue-free administration.

The solution in step a) comprises at least one hydrophobic polymer.

The term hydrophobicity refers to the property of a molecule to prefer either a polar or a non-polar (apolar) medium. The hydrophobicity, i.e. the water-repellent property of a molecule, increases as the non-polar character of the compound increases. Hydrophobic polymers have a correspondingly low hydrophilicity.

In a preferred embodiment, the at least one hydrophobic polymer has a log P value greater than about 1, preferably greater than about 1.5, and especially preferably greater than about 2.

The n-octanol-water partition coefficient K_{ow} (notations such as octanol/water partition coefficient are also common and correct) is a dimensionless partition coefficient known to a person skilled in the art which indicates the ratio of the concentrations of a chemical in a two-phase system of n-octanol and water and is thus a measure of the hydrophobicity or hydrophilicity of a substance. The log P value is the decadic logarithm of the n-octanol-water partition coefficient K_{ow}. The following is true:

$${K}_{o w} = P = \frac{{c}_{0}^{Si}}{{c}_{w}^{Si}}$$

$$and$$

$$log P = log \frac{{c}_{0}^{Si}}{{c}_{w}^{Si}} = log {c}_{0}^{Si} - {c}_{w}^{Si}$$

with c₀^{Si}=concentration of a chemical in the octanol-rich phase and
c_{w}^{Si}=concentration of a chemical in the water-rich phase.

K_{ow} is greater than one if a substance is more soluble in fat-like solvents such as n-octanol and less than one if it is more soluble in water. Accordingly, log P is positive for lipophilic substances and negative for hydrophilic substances.

In an especially preferred embodiment, the at least one hydrophobic polymer comprises cellulose acetate phthalate, hypromellose acetate succinate, poly(meth)acrylate, hypromellose acetate phthalate, polyvinyl acetate phthalate, ethyl cellulose, cellulose acetate butyrate, collophonium, polyoxyethylene alkyl ethers, polyvinyl acetate phthalate and/or cellulose nitrate.

The process according to the invention is further preferably characterised in that the hydrophilic polymer is added in an amount such that it constitutes from 30 to 99 wt. %, preferably from 35 to 90 wt. %, in relation to the total weight of the dried oral thin film.

The process according to the invention is further preferably characterised in that the hydrophobic polymer is added in an amount such that it constitutes from 1 to 70 wt. %, preferably from 10 to 65 wt. %, in relation to the total weight of the dried oral thin film.

The process according to the invention is further preferably characterised in that the hydrophobic pharmaceutically active ingredient is added in an amount such that it constitutes from 0.01 to 40 wt. %, preferably from 5 to 30 wt. %, in relation to the total weight of the dried oral thin film.

The hydrophobic pharmaceutically active ingredient preferably comprises a hydrophobic active ingredient having a log P value of greater than about 1, preferably greater than about 1.5, and especially preferably greater than about 2, as defined above.

The term “pharmaceutically active ingredient” also includes all pharmaceutically acceptable salts and solvates of the pharmaceutically active ingredient in question.

The hydrophobic active ingredient is preferably present substantially in the hydrophobic microparticles. The term “substantially” is understood to mean that the active ingredient is present in the microparticles of the oral thin film in a proportion of more than 85 wt. %, preferably more than 90 wt. % and especially preferably more than 95 wt. % or more than 99 wt. %, in relation to the total amount of active ingredient in the system.

Very especially preferably, the hydrophobic pharmaceutically active ingredient comprises idebenone, ulipristal acetate and/or ketoprofen.

Idebenone is understood to mean 2-(10-hydroxydecyl)-5,6-dimethoxy-3-methyl-cyclohexa-2,5-diene-1,4-dione, which is marketed for example under the trade name Raxone® by Santhera Pharmaceuticals.

Ulipristal acetate is understood to mean (8S,11S,13S,14R,17R)-17-acetoxy-11-[4-(dimethylamino)phenyl]-19-norpregna-4,9-diene-3,20-dione.

Ketoprofen is understood to mean (RS)-2-(3-benzoylphenyl)propionic acid.

Furthermore, flavourings and/or essential oils may preferably also be present in the microparticles.

The process according to the invention is additionally preferably characterised in that no emulsifier and/or pH regulator is added, leading to the result that no emulsifier and/or pH regulator is present in the obtained oral thin film comprising microparticles either.

Emulsifier is a term for auxiliary agents for the production and stabilisation of emulsions, which in a narrower sense can also be described as a surface-active substance or surfactant and is usually present as an oily to waxy, but also powdery substance.

All substances or mixtures of substances that have a buffering effect and thus serve to adjust or maintain a certain pH value are considered to be pH regulators.

The process according to the invention is additionally preferably characterised in that it further comprises the addition of at least one excipient selected from the group comprising colourants, flavourings, sweeteners, taste-masking agents, enhancers, humectants, preservatives and/or antioxidants to the solution a) and/or b), or b2) or the solvent from b1) and/or to the emulsion c), c1) or c2).

The process according to the invention is further preferably characterised in that these excipients are added in an amount such that they are each contained in an amount of from 0.01 to 25 wt. %, in relation to the total weight of the dried oral thin film.

The process according to the invention is additionally preferably characterised in that the emulsion from c), c1) or c2) is spread and dried such that the dried emulsion has a basis weight of from about 20 to about 250 g/m².

The process according to the invention is additionally preferably characterised in that the emulsion from c), c1) or c2) is spread and dried such that the dried emulsion has a layer thickness of from preferably about 10 μm to about 500 μm, preferably from about 50 μm to about 300 μm.

The emulsion is preferably spread with a squeegee.

Alternatively, a coating box, spiral doctor blade, slotted nozzle or roller applicator can be used for spreading.

The spread emulsion is preferably dried in a drying cabinet, alternatively in a climatic chamber, preferably for 10 min in a preferred temperature range of from 40° C. to 100° C.

The process according to the invention is additionally preferably characterised in that the emulsion from c), c1) or c2) is stirred or otherwise mixed. The strength of the stirring or mixing makes it possible to influence the size of the microparticles. In principle, the stronger the stirring or mixing, the smaller the resulting microparticles.

In one embodiment, a turbine stirrer is used to stir the emulsion. Suitable turbine stirrers are available, for example, from the companies IKA or Heidolph.

Alternatively, propeller stirrers, anchor stirrers, dissolvers, homogenisers, colloid mills, ultrasonic mixers or microfluidisers can be used for stirring or mixing.

The emulsion is preferably stirred or mixed for at least 15 minutes and at most 24 hours.

The emulsion is preferably stirred or mixed at a speed of at least 50 rpm and at most 8000 rpm.

The emulsion presented is preferably stirred or mixed at a temperature of from 4° C. to 50° C.

Preferably, the microparticles have a mean particle size of less than 100 μm, preferably less than 50 μm, preferably less than 20 μm, preferably less than 10 μm and very especially preferably less than 5 μm or less than 3 μm. The mean particle size is determined by light microscopy.

The present invention further relates to an oral thin film comprising microparticles obtainable by the process described above.

The definitions disclosed for the process according to the invention also apply to the oral thin film obtainable by the process described above.

An oral thin film comprising microparticles obtainable by the process is characterised especially in that the microparticles have a mean particle size of less than 100 μm, preferably less than 50 μm, preferably less than 20 μm, preferably less than 10 μm and very especially preferably less than 5 μm or less than 3 μm. The mean particle size is determined by light microscopy.

The advantage of such small microparticles lies in the increased bioavailability of the hydrophobic pharmaceutically active ingredient contained therein.

Lastly, the present invention relates to an oral thin film comprising microparticles obtainable by the process described above for use as a medicament, especially for the treatment of Friedreich's ataxia, Duchenne muscular dystrophy, primary progressive multiple sclerosis and/or Leber's hereditary optic neuropathy.

The invention will be explained below by means of non-limiting examples.

### EXAMPLES

### Example 1

TABLE 1

| Material | Function | Amount [wt. %] |
|---|---|---|
| Polyvinyl alcohol | Hydrophilic polymer | 68.85 |
| (PVA 4-88) | | |
| Hypromellose acetate | Hydrophobic polymer | 24.89 |
| succinate | | |
| Idebenone | Pharmaceutically | 6.24 |
| | active ingredient | |

For the production of an oral thin film according to the invention of the composition according to Table 1, 68.85 wt. % polyvinyl alcohol were dissolved in water.

In parallel, 24.89 wt. % hypromellose acetate succinate and 6.24 wt. % idebenone were dissolved in methyl acetate.

The polyvinyl alcohol solution and the solution comprising hypromellose acetate succinate and idebenone were then mixed to obtain an emulsion.

The emulsion obtained was spread out and dried.

The oral thin film obtained has a solid hydrophilic polymer matrix in which the hydrophobic microparticles are present as a separate stable phase. The microparticles have a mean particle size of less than 5 μm (determined by light microscopy).

### Example 2

TABLE 2

| | | Amount | Amount |
|---|---|---|---|
| | | [wt. %] | [wt. %] |
| | | in relation to | in relation |
| | | the dry | to the |
| Material | Function | constituents | solution |
| HPMC 603 | Hydrophilic | 38.5 | 15.4 |
| | polymer | | |
| HPMC 60SH60 | Hydrophilic | 0.75 | 0.30 |
| | polymer | | |
| Orange flavour | Flavouring | 3.00 | 1.20 |
| Mint flavour | Flavouring | 1.00 | 0.40 |
| Polysorbate 80 | Emulsifier | 3.00 | 1.20 |
| Span 85 | Emulsifier | 2.00 | 0.80 |
| Miglyol | Carrier Flavour- | 4.00 | 1.60 |
| | Phase | | |
| Sucralose | Sweetener | 1.00 | 0.40 |
| Saccharin sodium | Sweetener | 2.00 | 0.80 |
| BHT | Preservative | 0.25 | 0.10 |
| | Flavouring | | |
| Isomalt | Disintegrant | 10.00 | 4.00 |
| Eudragit | Hydrophobic | 9.50 | 3.80 |
| Smartseal | polymer | | |
| Ketoprofen | Pharmaceutically | 25.00 | 10.00 |
| | active ingredient | | |
| Water | Solvent | — | 45.00 |
| Methyl acetate | Solvent | — | 15.00 |

To produce an oral thin film according to the invention of the composition according to Table 2, 10.00 wt. % ketoprofen were dissolved in 15.00 wt. % hydrophobic solvent (methyl acetate). To this there were added 3.80 wt. % hydrophobic polymer (Eudragit Smartseal), and the mixture was stirred until the ketoprofen was dissolved.

To this solution there were added the hydrophilic polymers (Σ15.7%) according to Table 2.

Then 45% water was added as a hydrophilic solvent with rapid stirring to obtain an emulsion.

The remaining ingredients were added to this emulsion. HPMC 60SH50, isomalt, saccharin and sucralose were added one after the other. Separately, BHT, miglyol, Span 85, PS80, mint flavour and orange flavour were dissolved and added dropwise to the mass. Rinsing was performed with 1.314 g water and the mass was stirred at 2000 rpm for 5 min to degas. A homogeneous, milky emulsion was formed. The emulsion obtained was spread out and dried.

The oral thin film obtained has a solid hydrophilic polymer matrix in which the hydrophobic microparticles are present as a separate stable phase.

1. A process for producing an oral thin film comprising microparticles, comprising the steps of:
a) producing a solution comprising a hydrophobic polymer, a hydrophobic pharmaceutically active ingredient and hydrophobic solvent,
b1) providing a hydrophilic polymer and providing a hydrophilic solvent immiscible with the solvent from a), or
b2) providing a solution comprising a hydrophilic polymer and a hydrophilic solvent immiscible with the solvent from a),
c1) mixing the solution from a) with the hydrophilic polymer from b1) followed by the addition of the hydrophilic solvent from b1) immiscible with the solvent from a) to obtain an emulsion, or
c2) mixing the solution from a) and the solution from b2) to obtain an emulsion, and
d) spreading and drying the emulsion from c1) or c2) to obtain an oral thin film comprising microparticles.

2. The process according to claim 1, characterised in that the hydrophilic solvent is water or an aqueous solvent.

3. The process according to claim 1, characterised in that the hydrophobic solvent comprises ethyl acetate, methyl acetate, propyl acetate, liquid alkanes, liquid alkenes, aromatics, carboxylic acid esters, ethers and/or gasoline.

4. The process according to claim 1, characterised in that the hydrophilic polymer is selected from the group comprising agar-agar, gelatin and other gel-forming proteins, cellulose and derivatives thereof, alginic acid, galactomannan, carrageenan and other vegetable gums, pullulan, xanthan, pectin and other glucans, dextran, polyvinylpyrrolidone, polyvinyl alcohol, poly(meth)acrylates, polyalkylene glycols, carboxyvinyl polymers, polyethylene glycol-polyvinyl alcohol copolymers, shellac, polyvinyl caprolactam-polyvinyl acetate-polyethylene glycol graft and/or co-polymers thereof, chitosan, polyoxyethylene alkyl ether and/or co-polymers thereof.

5. The process according to claim 1, characterised in that the hydrophobic polymer has a log P value of greater than about 1.

6. The process according to claim 1, characterised in that the hydrophobic polymer comprises polymer cellulose acetate phthalate, hypromellose acetate succinate, poly(meth)acrylate, hypromellose acetate phthalate, polyvinyl acetate phthalate, ethyl cellulose, cellulose acetate butyrate, collophonium, polyoxyethylene alkyl ethers, polyvinyl acetate phthalate and/or cellulose nitrate.

7. The process according to claim 1, characterised in that the hydrophilic polymer is added in an amount such that it constitutes from 30 to 99 wt. % in relation to the total weight of the dried oral thin film.

8. The process according to claim 1, characterised in that the hydrophobic polymer is added in an amount such that it constitutes from 1 to 70 wt. % in relation to the total weight of the dried oral thin film.

9. The process according to claim 1, characterised in that the hydrophobic pharmaceutically active ingredient is added in an amount such that it constitutes 0.01 to 20 wt. % in relation to the total weight of the dried oral thin film.

10. The process according to claim 1, characterised in that no emulsifier and/or pH regulator is added.

11. The process according to claim 1, characterised in that the process further comprises the addition of at least one excipient selected from the group comprising colourants, flavourings, sweeteners, taste-masking agents, enhancers, humectants, preservatives and/or antioxidants to the solution a) and/or to the solution b) and/or to the emulsion c).

12. The process according to claim 1, characterised in that the hydrophobic pharmaceutically active ingredient comprises a pharmaceutically active ingredient having a log P of greater than 1.

13. The process according to claim 1, characterised in that the emulsion from c) is spread and dried so that the dried emulsion has a basis weight of about 20 to about 250 g/m².

14. An oral thin film obtainable by the process according to claim 1.

15. A method of delivering a pharmaceutically active ingredient comprising administrating the oral thin film according to claim 14 to a patient.
